(19) United States
(12) Patent Application Publication
(10) Pub. No.: US 2023/0360721 A1
(43) Pub. Date: Nov. 9, 2023

(54) IN SILICO PREDICTIONS OF AMYLOID-RELATED IMAGING ABNORMALITIES

(71) Applicant: HOFFMANN-LA ROCHE INC., LITTLE FALLS, NJ (US)

(72) Inventors: Roxana Aldea, Basel (CH); Hans Peter Grimm, Basel (CH); Norman A. Mazer, Pfeffingen (CH)

(73) Assignee: HOFFMANN-LA ROCHE INC., LITTLE FALLS, NJ (US)

(21) Appl. No.: 18/140,583

(22) Filed: Apr. 27, 2023

Related U.S. Application Data
(60) Provisional application No. 63/337,992, filed on May 3, 2022.

(51) Int. Cl. G16B 5/20; G16B 40/20; G06T 7/00; G16H 50/20

(52) U.S. Cl. CPC G16B 5/20; G16B 40/20; G06T 7/0012; G16H 50/20; G06T 2207/20076

(57) ABSTRACT

A dosage of an active ingredient in a treatment that is being provided to or that is being considered for provision to a subject is identified. A level of local amyloid beta is predicted based on the dosage of the active ingredient. A severity of amyloid-related imaging abnormalities (ARIA) manifested as hyperintensities on T₂-weighted fluid attenuated inversion recovery (FLAIR) images (ARIA-E) is predicted based on the predicted removal of local amyloid beta, where predicting severity of ARIA-E includes predicting an extent of vascular wall disturbance. A result corresponding to the predicted ARIA-E severity is output.

### CROSS-REFERENCES TO RELATED APPLICATIONS

The application claims the benefit of and the priority to U.S. Provisional Application No. 63/337,992, filed May 3, 2022, which is hereby incorporated by reference in its entirety for all purposes.

### BACKGROUND

Over the last decades, amyloid-beta (Aβ) aggregates have been the most common target of clinical trials investigating disease-modifying therapies in Alzheimer's disease. Several therapies based on monoclonal antibodies against Aβ aggregates reported amyloid-related imaging abnormalities (ARIA). Two ARIA types have been identified by magnetic resonance imaging (MRI) of the brain. ARIA-E, manifested as hyperintensities on T2-weighted fluid attenuated inversion recovery (FLAIR) images, are suggestive of vasogenic edema, sulcal effusion and gyral swelling. ARIA-H, manifested as hypointensities on T2*-weighted gradient echo sequences, are thought to represent hemosiderosis and microhemorrhages. ARIA-E is transient and typically resolves within months, whereas ARIA-H remains visible on subsequent MRIs. As assessed with various radiological scales that show good correlation, most ARIA-E events are mild to moderate in severity and are usually not associated with symptoms.

The risk of therapy-related ARIA appears to be dose-dependent and increases in APOE ε4 carriers. In order to mitigate the risk of ARIA, several anti-amyloid clinical trials implemented dose escalation schemes towards the target dose, as well as routine brain MRI monitoring. The incidence of therapy-related ARIA events across various anti-amyloid clinical trials has been reported within the 10-42% range, with the highest rates occurring in the APOE ε4 allele carriers. Spontaneous ARIA events have rarely been seen in the placebo arm (e.g. <3% ARIA-E) and outside the setting of anti-amyloid clinical trials. An example of the latter are the ARIA-like events occurring in cerebral amyloid angiopathy-related inflammation (CAA-ri), a rare autoimmune reaction against vascular Aβ aggregates. In clinical practice, ARIA can become a high burden for patients with Alzheimer's disease and healthcare systems due to MRI monitoring requirements. Moreover, ARIA may limit the ability of patients to reach the target dose and to benefit from amyloid-lowering disease modifying therapies.

Therefore, it would be advantageous to be able to generate predictions of therapy related ARIA events and their severity.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

The patent or application file contains at least one drawing executed in color. Copies of this patent or patent application publication with color drawing(s) will be provided by the Office upon request and payment of the necessary fee.

The present disclosure is described in conjunction with the appended figures:

FIG. 1 depicts the main pathophysiological pathways connecting the drug-mediated removal of Aβ aggregates to ARIA.

FIG. 2 depicts an exemplary ARIA-E prediction network that includes an ARIA-E prediction system and a user device.

FIG. 3 illustrates a vascular wall disturbance (VWD) model and related illustrative time-course and variable-relationship plots.

FIG. 4 illustrates exemplary model-performance data at a subject level.

FIG. 5 shows a flowchart of an exemplary process 400 for predicting ARIA-E according to some embodiments of the invention.

FIG. 6 shows a scatter plot of exemplary residuals generated by the PK model used to predict the drug concentration in plasma for the ARIA-E cases from the SR/MR OLE studies. Individual weighted residuals (IWRES) are shown. The solid lines represent the empirical (10th, 50th and 90th) percentiles, while the dotted lines represent the predicted (10th, 50th and 90th) percentiles.

FIG. 7 shows scatter plots of exemplary residuals generated by part one (top panel) and part two (bottom panel) of the two-part PD model used to predict the probability of ARIA-E occurrence and the ARIA-E magnitude, respectively. Normalized prediction distribution errors (NPDE) and IWRES are also shown. The solid lines represent the empirical (10th, 50th and 90th) percentiles, while the dotted lines represent the predicted (10th, 50th and 90th) percentiles.

FIGS. 8A-8C shows data that illustrates the effect of model parameters on amyloid-related imaging abnormalities with edema/effusion (ARIA-E) dynamics.

### SUMMARY

In some embodiments, a computer-implemented method is provided. The method includes dosage of an active ingredient in a treatment that is being provided to or that is being considered for provision to a subject. The method further includes predicting a level of local amyloid beta based on the dosage of the active ingredient and predicting a severity of amyloid-related imaging abnormalities (ARIA) manifested as hyperintensities on T₂-weighted fluid attenuated inversion recovery (FLAIR) images (ARIA-E) based on the predicted removal of local amyloid beta, where predicting severity of ARIA-E includes predicting an extent of vascular wall disturbance. The method also includes outputting a result corresponding to the predicted ARIA-E severity

The active ingredient may include an anti-amyloid monoclonal antibody.

The method may include using a pharmacokinetic model to predict a time course of a concentration of an active ingredient in the treatment in plasma, where the prediction of the local amyloid beta is based on at least one predicted concentration of the active ingredient in the predicted time course.

Predicting the level of local amyloid beta may include estimating a baseline level of local amyloid beta; calculating a rate of removal of the local amyloid beta based on the dosage of an active ingredient and the baseline level of local amyloid beta; and predicting the level of local amyloid beta based on the calculated rate of removal of the local amyloid beta.

Predicting the level of local amyloid beta may include solving a pharmacodynamic differential equation that assumes a rate of change of amyloid beta is proportional to a product between concentrations of the active ingredient in the subject and local amyloid beta levels.

Predicting the level of vascular wall disturbance may include solving a differential equation that includes the level of local amyloid beta.

Predicting the severity of ARIA-E may include solving an algebraic equation that assumes a non-linear relationship between the level of vascular wall disturbance and the Barkhof Grand Total Score (BGTS).

The method may include identifying a potential schedule for monitoring for ARIA events based on the predicted severity of ARIA, where the result characterizes the potential schedule.

The method may include identifying a potential recommendation of the dosage of the active ingredient for the subject based on the predicted severity of ARIA, where the result characterizes the potential recommendation dosage.

In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions that have been employed are used as terms of description and not of limitation, and there is no intention in the use of such terms and expressions of excluding any equivalents of the features shown and described or portions thereof. It is recognized, however, that various modifications are possible within the scope of the systems and methods claimed. Thus, it should be understood that, although the present system and methods have been specifically disclosed by examples and optional features, modification and variation of the concepts herein disclosed should be recognized by those skilled in the art, and that such modifications and variations are considered to be within the scope of the systems and methods as defined by the appended claims.

This summary is not intended to identify key or essential features of the claimed subject matter, nor is it intended to be used in isolation to determine the scope of the claimed subject matter. These illustrative examples are mentioned not to limit or define the disclosure, but to provide examples to aid understanding thereof. Additional embodiments and examples are discussed in the Detailed Description, and further description is provided there. The subject matter should be understood by reference to appropriate portions of the entire specification of this disclosure, any or all drawings, and each claim.

The foregoing, together with other features and embodiments will become more apparent upon referring to the following specification, claims, and accompanying drawings.

### DETAILED DESCRIPTION

The exact pathophysiological mechanisms of ARIA remain to be fully elucidated. It has been proposed that the drug-mediated removal of Aβ aggregates can increase the permeability of the cerebrovascular wall to the entrance of fluid and blood products into the brain, leading to ARIA-E and ARIA-H, respectively. In the Alzheimer's brain, the Aβ aggregates are found in the brain parenchyma, as well as in the wall of cerebral blood vessels in the form of cerebral amyloid angiopathy (CAA). The anti-amyloid antibodies associated with ARIA bind various forms of Aβ aggregates (e.g. oligomers, fibrils and plaques) and remove them by dissolution and/or through effector cell-mediated phagocytosis.

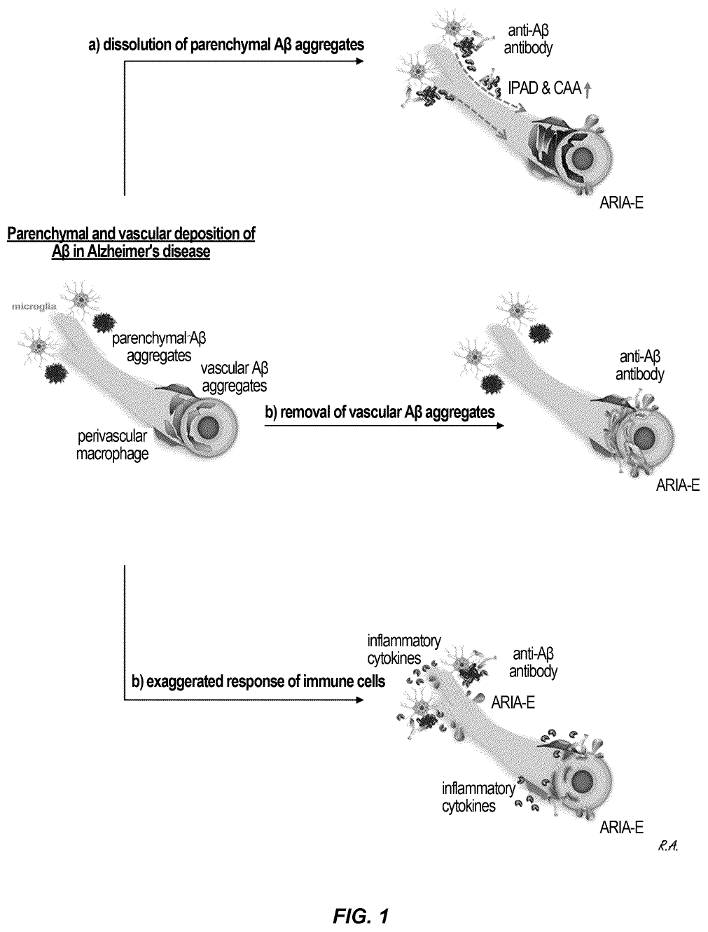

FIG. 1 depicts the main pathophysiological pathways thought to connect the drug mediated removal of Aβ aggregates to ARIA. Overall, it appears that all postulated pathomechanisms of ARIA include some degree of disturbance in the cerebrovascular wall. It remains unclear to what extent the degree of amyloid burden, or the location and rate of amyloid clearance, influence the onset, time course and severity of ARIA. Gaining a better understanding of ARIA etiology has the potential to unveil additional risk factors that could accelerate the path towards patient-centric strategies for dosing and monitoring in individuals with Alzheimer's disease.

Some embodiments of the present invention relate to predicting temporal dynamics and dose-dependency of ARIA-E severity by using a semi-mechanistic, in silico model of ARIA-E, referred to as the Vascular Wall Disturbance (VWD) model. For example, the model may receive input data that includes dosage information that indicates an amount of anti-amyloid monoclonal antibodies (e.g. gantenerumab) that is to be administered in the simulation in accordance with a given schedule (e.g., such that the model simulates administration of the dosage every four weeks), and the model may generate a result corresponding to a predicted occurrence and/or severity of ARIA (or ARIA-E) at each of a set of time points relative to a reference point in the schedule (e.g., administration of a first dosage). The predicted severity can be used to generate a potential monitoring schedule, dosage schedule, and/or dosage-adjustment criteria. For example, based on an initial dosage plan, a schedule for monitoring and safety data collection may be proposed, and a dosage-adjustment criteria may be defined to propose tapering a dosage by a defined amount only if an MRI shows at least a defined extent of ARIA-E.

The framework of the VWD model allows different biological interpretations of the modeled mechanisms, in line with the ARIA-E pathomechanisms proposed in FIG. 1. The VWD model links several pharmacological and biological factors to the observed drug pharmacokinetics and magnitude of ARIA-E. One biological factor considered in the model is the local amyloid, either vascular or parenchymal, whose drug-mediated removal may trigger a cascade of events leading to ARIA-E in one or more regions of the brain. Another biological variable of the model is the level of VWD, which is a hypothetical measure of susceptibility to fluid leakage into the brain due to disrupted vascular integrity and/or perivascular inflammation induced by the amyloid-drug interaction. The VWD model configuration allows assessment of the potential interplay between drug-mediated removal of local amyloid and intrinsic vascular repair processes, which can, determine or influence the level of VWD, which further influences the magnitude of ARIA-E.

### ARIA-E Prediction Network

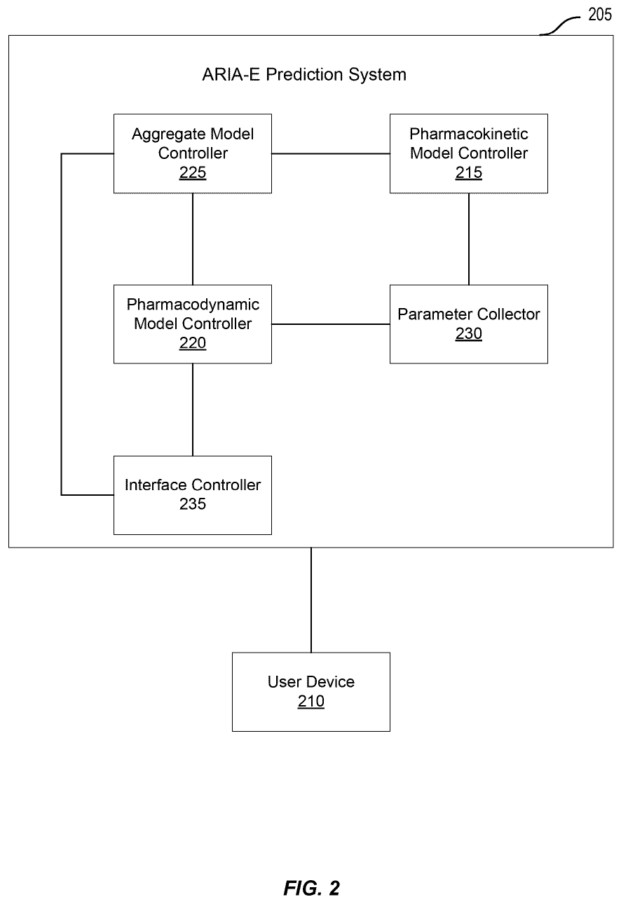

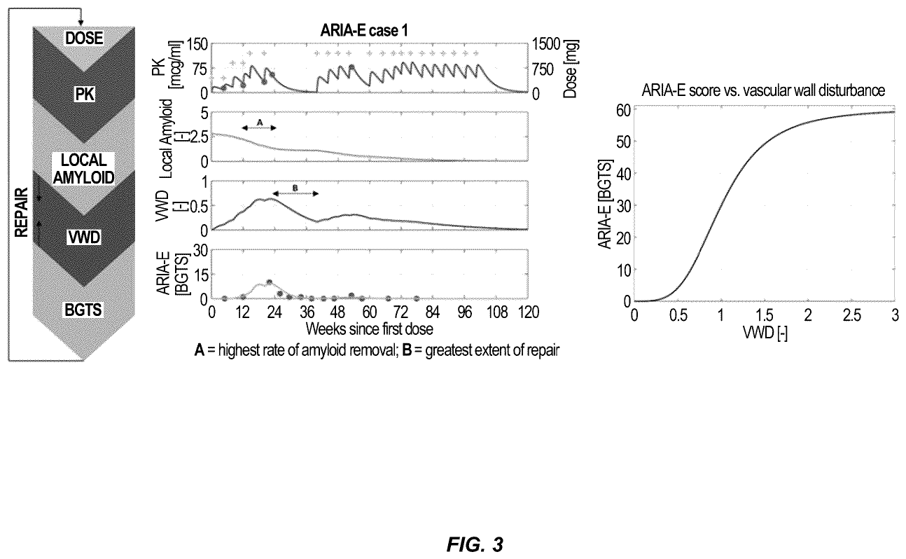

FIG. 2 depicts an ARIA-E prediction network that includes an ARIA-E prediction system 205 and a user device 210. ARIA-E prediction system 205 controls a VWD model, which includes both a pharmacokinetic model component (controlled by a pharmacokinetic model controller 215) and a pharmacodynamic model component (controlled by a pharmacokinetic model controller 220). The VWD model (as represented in the left side of FIG. 3) can be a semi-mechanistic pharmacokinetic pharmacodynamic model that integrates know and/or presumed processes and/or that (e.g., at the same time) accounts for inter-individual variability of model parameters.

Each of pharmacokinetic model controller 215 and pharmacodynamic model controller 220 can be configured to specify equations used in a model, to retrieve stored data (e.g., parameter values) from memory, and to execute the respective model components. An aggregate model controller 225 may call, send input data to, and/or may receive outputs from pharmacokinetic model controller 215 and/or pharmacodynamic model controller 220. Thus, aggregate model controller 225 may be configured to control the definition of and execution of the VWD model by interacting with pharmacokinetic model controller 215 and/or pharmacodynamic model controller 220. Aggregate model controller 225 may further perform pre-processing and/or post-processing. For example, such pre-processing and/or post-processing may adjust for subject-specific characteristics, such as age, APOE e4 genotype, baseline ARIA-H load and vascular risk factors (which may alternatively or additionally be factors that are used by the pharmacokinetic and/or pharmacodynamic models to generate predictions).

The pharmacokinetic model component is configured to predict a time course of the concentration of an active ingredient in plasma of a subject based on dosing history and longitudinal observations of concentration in plasma. An exemplary model can include a pharmacokinetics model disclose by Retout S, et al. “Disease Modeling and Model-Based Meta-Analyses to Define a New Direction for a Phase III Program of Gantenerumab in Alzheimer's Disease.” Clin Pharmacol Ther. 2022 April; 111(4):857-866, which is hereby incorporated by reference in its entirety for all purposes.

The top graph in the middle panel of FIG. 3 shows an exemplary time course of a concentration of an active ingredient in a subject's plasma (left axis), as estimated by using a pharmacokinetics model, when the active ingredient was administered at the time points indicated by the horizontal positions of the plus signs at doses as indicated by the right-axis vertical positions of the plus signs.

The pharmacodynamic model component is configured to predict ARIA-E observations based on concentrations of an active ingredient based on intermediate calculations that predict changes in levels of local amyloid-β and vascular wall disturbance triggered based on the active ingredient. The intermediate calculations may include solving one or more differential equations and/or performing one or more algebraic calculations. Exemplary equations that can be used in the pharmacodynamic model component follow.

A first exemplary differential equation is based on an assumption that a rate of drug-mediated removal of local Aβ depends on the product of drug concentration in plasma

$$Cp ( t ) [ \frac{mcg}{m l} ]$$

and the level of local Aβ(t) [arbitrary units] via the parameter

$${α}_{removal} [ {( day \cdot \frac{mcg}{ml} )}^{- 1} ] : \frac{dA β ( t )}{dt} = - {α}_{removal} \cdot Cp ( t ) \cdot A β ( t ) , \qquad ( 1 )$$

where t is the time and the initial value of Aβ at the start of the (open label extension) OLE treatment (i.e. Aβ(t=0)) is Amyloid₀. For a given model execution, the initial value of Aβ (which way be parenchymal or vascular Aβ) may be estimated or provided, depending on availability of data.

Notably, this equation refers to the local level of the modeled amyloid, which represents a hypothetical measure of either the vascular or the parenchymal arnyloid from the brain regions, being affected by ARIA-E. The removal rate of this ‘Local Amyloid’ is assumed proportional to both drug concentration in plasma and existing local amyloid level. The proportionality factor, here denoted αᵣₑₘₒᵥₐₗ and referred to as the removal constant, could be interpreted as the rate constant of the biochemical reaction between anti-amyloid antibodies and amyloid aggregates. Such a reaction corresponds to the drug-mediated amyloid removal to occur and its magnitude may vary across individuals. The baseline level of local amyloid (here denoted Amyloid₀) impacts the initial rate of arnyloid removal and may also vary across individuals. Other processes that could alter the local amyloid level, such as physiological amyloid production and endogenous amyloid clearance, are not included in the model.

The second-to-top graph in the middle panel in FIG. 3 illustrates an estimated time course of local Aβ, as estimated by using Eqn. (1). In this particular instance, it is estimated that the local amyloid decreases over time.

A second exemplary differential equation assumes that the removal of local Aβ drives the build-up of VWD(t) [arbitrary units], which is counter-acted by a first-order vascular repair process characterized by the repair rate kᵣₑₚₐᵢᵣ [day⁻¹]):

$$\frac{dVWD ( t )}{dt} = {α}_{removal} \cdot Cp ( t ) \cdot A β ( t ) - {k}_{repair} \cdot VWD ( t ) , \qquad ( 2 )$$

where the initial value of VWD at the start of the OLE treatment (i.e. VWD(t=0)) is zero.

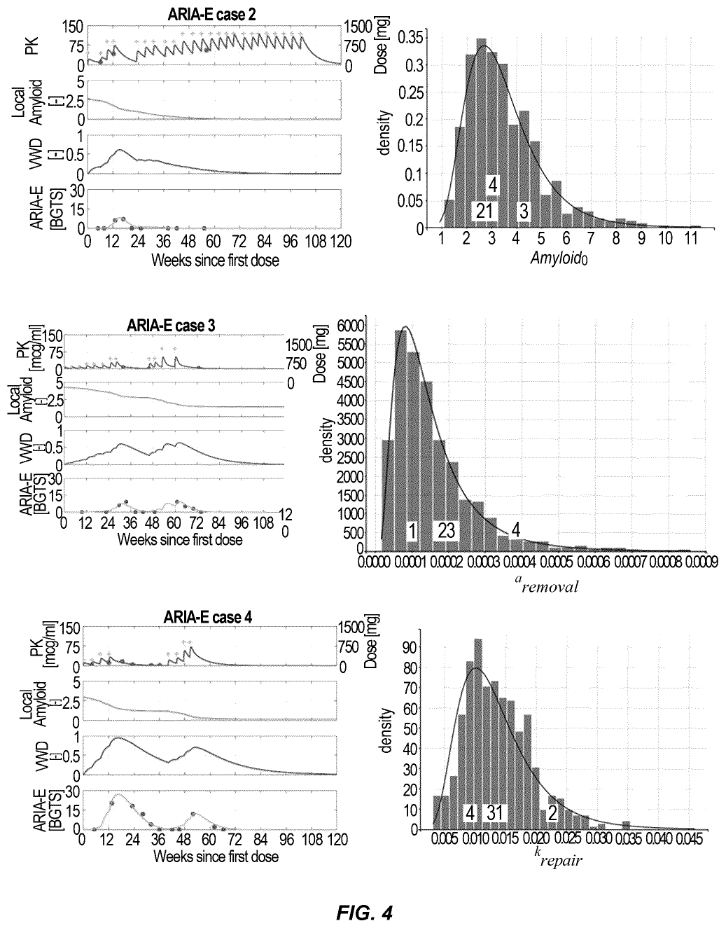

The parameters Amyloid₀, αᵣₑₘₒᵥₐₗ and kᵣₑₚₐᵢᵣ are allowed to vary across subjects, their statistical distributions (typical value and width) being estimated, as illustrated in FIG. 4. One, more or all of the remaining model parameters may be constant across individuals.

The third-to-top graph in the middle panel of FIG. 3 illustrates an estimated time course of VWD, as estimated by using Eqn. (2). In this particular instance, it is estimated that VWD first increases and then generally decreases.

The ‘VWD’ variable corresponds to a hypothetical concept that describes the susceptibility to fluid leakage into the brain due to therapy-induced defects in the vascular and perivascular structures of the brain, such as impaired blood-brain barrier and burdened intramural periarterial drainage pathways. In the absence of any disturbance, VWD is equal to zero. VWD buildup is driven by drug-mediated amyloid removal and is counteracted by a presumed intrinsic vascular repair process. With regard to Eqn. (2), the vascular repair is assumed to be a first-order process with a rate constant (kᵣₑₚₐᵢᵣ) and half-life independent of the VWD magnitude. The VWD magnitude may be related to the predicted ARIA-E score (BGTS) according to (for example) a sigmoidal function. This relationship can ensure zero or small BGTS at low levels of VWD, a sharp rise in ARIA-E score at intermediate levels and imposes an upper limit for ARIA-E magnitude (BGTS=60) at high levels of VWD.

Two additional exemplary algebraic equations connect VWD to the observed ARIA-E magnitude. Using two equations for this connection addresses a complication that a large number of zero BGTS values exist in the dataset of ARIA-E scores, where a first equation predicts a probability of an ARIA-E event (i.e. a non-zero BGTS) for each BGTS observation, while a second equation quantifies the magnitude of ARIA-E once an event was predicted to occur with high probability.

The first exemplary algebraic equation deals with the probability p(t) of a positive (non-zero) BGTS under the assumption that the logarithm of the odds ratio is linearly related to VWD(t) by

$$ln ( \frac{p ( t )}{1 - p ( t )} ) = {β}_{1} \cdot ( VWD ( t ) - {VWD}_{50} ) , \qquad ( 3 )$$

where β₁ denotes the linear factor and the parameter VWD₅₀ represents the value of VWD(t) that generates a 50% probability of a positive BGTS. The second exemplary algebraic equation quantifies the BGTS magnitude by using the sigmoidal response function

$$BGTS ( G ) = {BGTS}_{max} \cdot \frac{{( \frac{VWD ( t )}{EG 50} )}^{pow}}{1 + {( \frac{VWD ( t )}{EG 50} )}^{pow}} , \qquad ( 4 )$$

where BGTSₘₐₓ represents the maximum ARIA-E score and EG50 [arbitrary units] represents the value of VWD(t) leading to half-maximal BGTS. Moreover, pow is the slope factor that gives the sensitivity of response to the VWD range by determining the steepness of the VWD-BGTS curve. Notably, Eqns. (3) and (4) use different types of BGTS data. The sigmoidal graph on the right side of FIG. 3 illustrates an exemplary relationship corresponding to Eqn. (4). In Eqn. (3), all the BGTS observations are transformed to Boolean data that indicates whether the observed BGTS is zero or positive, while for Eqn. (4), the BGTS observations are restricted to the strictly positive values.

The bottom graph in the middle panel of FIG. 3 illustrates an estimated time course of BGTS for the exemplary instance pertaining to the other graphs in the middle panel. The estimated time course of BGTS is determined using Eqns. (3) and (4). In this exemplary instance, the BGTS increases shortly after the beginning of the treatment regimen and then generally decreases.

Aggregate model controller 225 may use the predicted BGTS to facilitate determining a proposed dosage schedule and/or proposed monitoring schedule for individuals that experienced ARIA-E. For example, aggregate model controller may coordinate generating predicted time courses of BGTS for a variety of prescribed schedules for dosing, for a variety of prescribed schedules for monitoring, and/or for a variety of different BGTS cut-off values (thresholds of ARIA-E severity that—in the model—would result in treatment interruption). Therefore, for each prescribed dosing schedule, monitoring schedule, and/or BGTS cut-off value, a predicted progression of ARIA-E severity (time course of BGTS) can be generated, which may subsequently be output (e.g., via interface controller 235) to aid in a user's (e.g., clinician) decision as to which dosing schedule, monitoring schedule, and/or BGTS cut-off value to use in a clinical study and/or a real-world use case.

Thus, the VWD model can interconnect the following factors: (i) gantenerumab doses, (ii) drug concentration in plasma, (iii) drug-induced removal of local amyloid, (iv) VWD presumed to lead to fluid leakage into the brain, (v) an intrinsic vascular repair process and (vi) the BGTS that quantifies the severity of ARIA-E. Individual-specific information about the given dose and time of administration can be provided as input, while the parameters related to some or all the other factors can be estimated.

A parameter collector 230 initially identifies parameters of each of the pharmacokinetic model component and the pharmacodynamic model component. In some instances, parameter collector 230 identifies a value for each of one or more parameters by querying an external source for the parameter. In some instances, parameter controller 230 learns a value for each of one or more parameters by fitting a model component or the VWD model using one or more training data sets. For example, a training data set may include results from a study where a set of subjects (e.g., each of whom had been diagnosed with Alzheimer's disease) receive a given active ingredient (or active ingredients corresponding to a particular class of active ingredients), such as gantenerumab, at one or more particular dosages and in accordance with one or more dosage schedules. A dosage schedule may indicate relative times at which various dosages of the active ingredient are administered relative to (for example) an initial dosage, a most-recent dosage, or another baseline. In some instances, the dosage of the active ingredient administered at each identified administration times in the schedule is the same relative to each other administration time. In some instances, the dosages of the active ingredient administered at each of at least two of the administration times are different. For example, the dosages may ramp up over at least two of the dosages. The study may have been conducted such that MRIs were regularly conducted and evaluated to monitor for ARIA-E and such that samples (e.g., blood samples) were regularly collected and evaluated to determine concentrations of an active ingredient. The evaluation may include assigning a severity to any detected ARIA-E, using a scoring technique such as the Barkhof Grand Total Score.

Study data from a data source may further be filtered to selectively include data corresponding to a subset of subjects, such that the training data set includes the selective data. The subset of subjects may (for example) include those for whom an ARIA-E occurrence (e.g., corresponding to a severity greater than a given threshold, such as a severity greater than zero) was observed during the study or within a year from a first dosage in the study.

In some instances, parameter collector 230 identifies parameters of a single model component in isolation of the other model component. For example, parameters for the pharmacokinetic model components may be defined by performing a Bayesian analysis on study data.

In some instances, parameter collector 230 fixes some parameters while fitting other parameters. For example, parameter collector 230 may fix parameters of the pharmacokinetic model component while fitting the pharmacodynamic model component. Parameter collector 230 may estimate parameter values using a nonlinear mixed effects modeling approach.

Parameter collector 230 can use assumptions about distributions of variables (e.g., a shape of a distribution), a maximum value, whether various variables depend on each other, etc. For example, parameter controller 225 can define an assumption that parameters Amyloid₀, αᵣₑₘₒᵥₐₗ and kᵣₑₚₐᵢᵣ have log-normal inter-individual variability, estimate their typical value efixed effect), and estimate the width of the distribution (random effect). As another example, Parameter collector 230 can define estimates of parameters β₁, VWD₅₀ and pow without any inter-individual variability (i.e. as fixed effects only). It can be shown that VWD(t) is proportional to Amyloid₀, implying that the parameters Amyloid₀ and EG₅₀ effectively appear only as a ratio in the expression for BGTS(t). To ensure structural identifiability, parameter collector 230 can set EG₅₀ to one, without any loss of generality. Parameter collector 230 can fix a maximum value of BGTS to 60, in line with the limit of the 60-point BGTS severity scale. The residual (unexplained) variability that describes the difference between observed and predicted BGTS can be assumed to be independent of the predictions and to be normally distributed.

ARIA-E prediction system 205 includes an interface controller 235 that facilitates communications between ARIA-E prediction system 205 and user device 210. Interface controller 235 may be configured to process incoming communications from user device 210 to (for example) detect a request for predictions generated by aggregate model controller (e.g., based on outputs from pharmacokinetic model controller 215 and/or pharmacodynamic model controller 220) and/or to detect information to be used as an input for the prediction (e.g., identifying a dosage and/or dosage schedule used to treat a given subject or being considered for treatment of a given subject).

Interface controller 235 may further or alternatively be configured to generate code that can be read by a software application and can trigger generation of a presentation or interface (e.g., a webpage), which may include or represent one or more predictions generated by ARIA-E prediction system 205. For example, interface controller 235 may generate webpage code configured to, when executed by a browser, present a webpage (on a website) that identifies the one or more predictions. The website and/or webpage may have initially also have operated to have collect input at user device 210 that was used to execute the model.

When user device 210 receives the information and/or code from ARIA-E prediction system 205, user device 210 may generate and present a presentation that includes the information and/or data that the code specifies is to be presented. User device 210 may thus be presenting data corresponding to predictions as to whether or an extent to which a given treatment regimen may result in ARIA-E.

### ARIA-E Prediction Process

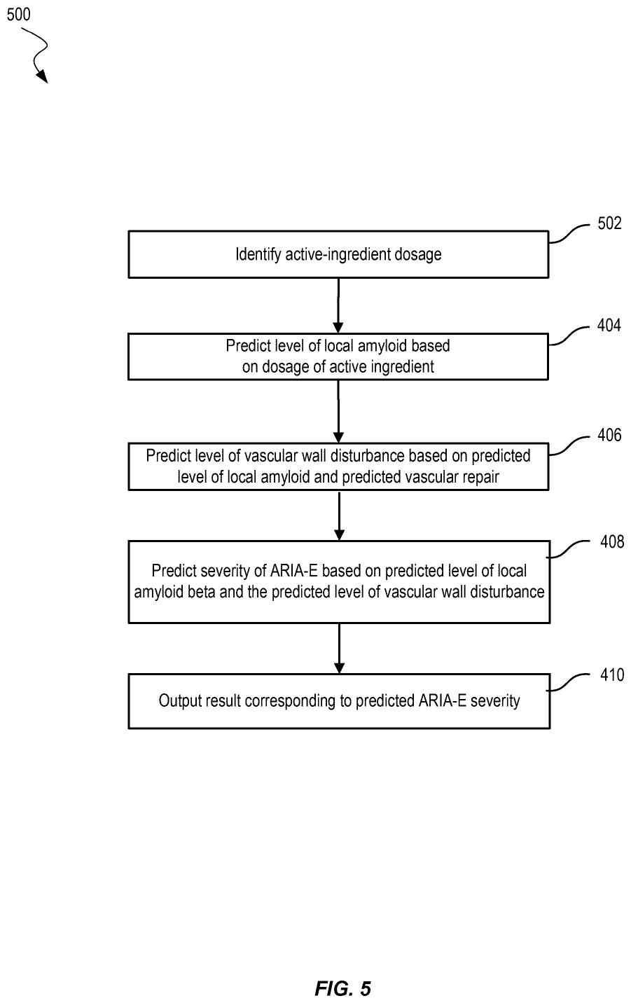

FIG. 5 shows a flowchart of an exemplary process 500 for predicting ARIA-E according to some embodiments of the invention. At block 502, aggregate model controller 225 identifies an actual or potential dosage of an active ingredient The identification may include identifying an actual or potential dosage that was or that is to be administered at a given time within a treatment regimen (e.g., time zero, 8 weeks after a start of the regimen, once a steady dose is reached, etc. The dosage may be (for example) a dosage indicated in a communication received from user device 210 (e.g., as identified by interface controller 235), a default dosage (e.g., as identified and/or retrieved by aggregate model controller 225), one or multiple potential dosages (e.g., as identified and/or retrieved by aggregate model controller 225), etc.

At block 504, pharmacodynamic model controller 220 predicts a level of local amyloid. More specifically, pharmacokinetic model controller 125 can predict a time course of a concentration of an active ingredient based on the dosage of the active ingredient, and pharmacodynamic model controller 220 predicts a time course of the level of local amyloid based on the predicted concentration of the active ingredient. The predicted level of local amyloid may be predicted at a same time that the dosage was administered, predicted on a date at a particular time relative to when a first (or a given) dosage was administered, predicted for each of multiple time points relative to when a first (or a given) dosage was administered, etc.

At block 506, pharmacodynamic model controller 220 predicts a level of vascular wall disturbance based on the predicted level of local amyloid and a predicted vascular repair. The predicted level of vascular wall disturbance may be generated by solving a differential equation, such as Eqn. (2). The predicted level of vascular wall disturbance may further depend on (for example) a predicted repair rate.

At block 508, pharmacodynamic model controller 220 predicts ARIA-E severity at one or multiple times (e.g., a time course of ARIA-E severity). Block 408 may include predicting the level(s) and/or using predicted levels of local amyloid beta at one or multiple time points (e.g., a time course of levels). Block 506 may include predicting the level(s) of and/or using predicted levels of vascular wall disturbance at one or multiple time points (e.g., a time-course of vascular wall disturbance). The local amyloid beta levels may be predicted using one or more differential equations and based on estimated active-ingredient concentration levels. The extents of vascular wall disturbance may be predicted using one or more differential equations and based on the predicted local amyloid beta levels (e.g., and/or using an approach as indicated with reference to block 406).

At block 510, a result can be output that corresponds to the predicted ARIA-E severity levels. For example, ARIA-E prediction system can transmit the result to user device 210. The result may include one or more predicted ARIA levels (e.g., a time course of ARIA-E levels), a potential schedule for monitoring for MRI-based monitoring of ARIA-E (e.g., via BGTS scoring), or a potential revised dosage schedule (e.g., that indicates one or more proposed dosages of an active ingredient and/or relative timing between one or more sequential dosage administrations).

### EXAMPLES

### Dynamical Behavior of Illustrative Cases

The data used for model development consists of longitudinal measurements of drug concentration in plasma and of ARIA-E magnitude from a subset of individuals (N=112) who developed ARIA-E during the open label extensions (OLE) of the Scarlet Road (SR; NCT01224106) and Marguerite Road (MR; NCT02051608) studies of gantenerumab, which included participants with prodromal Alzheimer's disease and mild Alzheimer's disease studies, respectively. Briefly, the SR/MR study participants that received the double-blind treatment and had at least one follow-up visit were eligible for open-label extension (OLE) participation. During the SR/MR OLE studies, all participants received subcutaneous gantenerumab every four weeks, with gradual up-titration towards the target dose of 1200 mg. Each participant was assigned to 1 of 5 dose titration regimens based on their APOE4 carrier status and the last dose during the double-blind treatment (Table 1). Protocol-defined routine MRI monitoring for ARIA-E detection was performed at regular intervals. If ARIA-E was detected, dosing was adjusted based on its radiological severity and/or presence of symptoms. The magnitude of ARIA-E on MRI scans was assessed with the Barkhof Grand Total Score (BGTS), a 60-point severity scale where higher scores indicate greater severity. The reported BGTS accounted for the number and size of parenchymal hyperintensities, sulcal hyperintensities and gyral swelling present on FLAIR images.

TABLE 1
Up-titration regimens for OLE in the SR and MR phase III trials

| | | | | | | | Week | Week | Week |
|---|---|---|---|---|---|---|---|---|---|
| # | Day 1 | Week 4 | Week 8 | Week 12 | Week 16 | Week 20 | 24-28 | 32-36 | 40-100 |
| 1 | 105 | 105 | 105 | 225 | 225 | 225 | 450 | 900 | 1200 |
| 2 | 225 | 225 | 450 | 450 | 900 | 900 | 1200 | 1200 | 1200 |
| 3 | 300 | 300 | 600 | 600 | 1200 | 1200 | 1200 | 1200 | 1200 |
| 4 | 450 | 450 | 900 | 900 | 1200 | 1200 | 1200 | 1200 | 1200 |
| 5 | 600 | 600 | 1200 | 1200 | 1200 | 1200 | 1200 | 1200 | 1200 |

All the doses shown for the five regimens are in milligrams (mg).

The four ARIA-E cases, illustrated in FIG. 3 and FIG. 4, were selected in order to highlight a variety of ARIA-E features (magnitude, resolution, recurrence) that are mechanistically insightful, but do not reflect the typical ARIA-E profile of gantenerumab. Each study participant was assigned to 1 of 5 dose titration regimens described in Table 1. Since the model accounted for inter-individual variability, the modeled temporal profiles of each ARIA-E case were generated based on empirical Bayes estimates, i.e. the most probable values of the individual parameters.

The VWD parameters were estimated by fitting the full VWD model to the collective set of longitudinal BGTS observations, while keeping the individual pharmacokinetic parameters fixed. The Boolean data from the first part (e.g. YES/NO for ARIA-E occurrence) and the continuous observations from the second part (e.g. the BGTS values within the range for ARIA-E magnitude) were implemented in the data file as two distinct observation types and modelled simultaneously. The parameter estimation was conducted with the nonlinear mixed effects method from Monolix (version 2020R1).

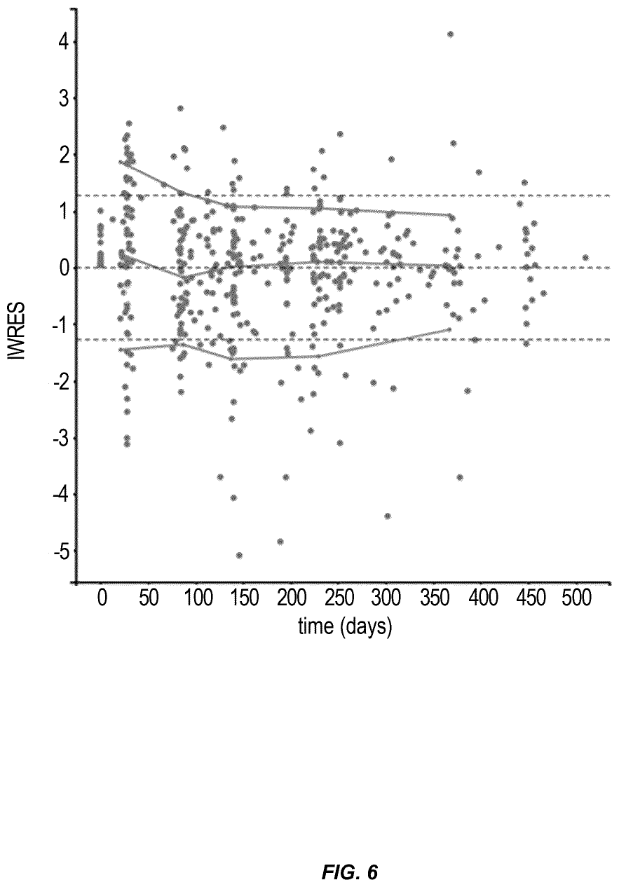

The data shown in FIG. 6 indicates that this previously built population pharmacokinetic model was able to describe the individual PK data from the SR/MR OLE studies: the residuals are evenly distributed around zero with most values within −2 and +2 standard deviations, thereby indicating no major systemic bias.

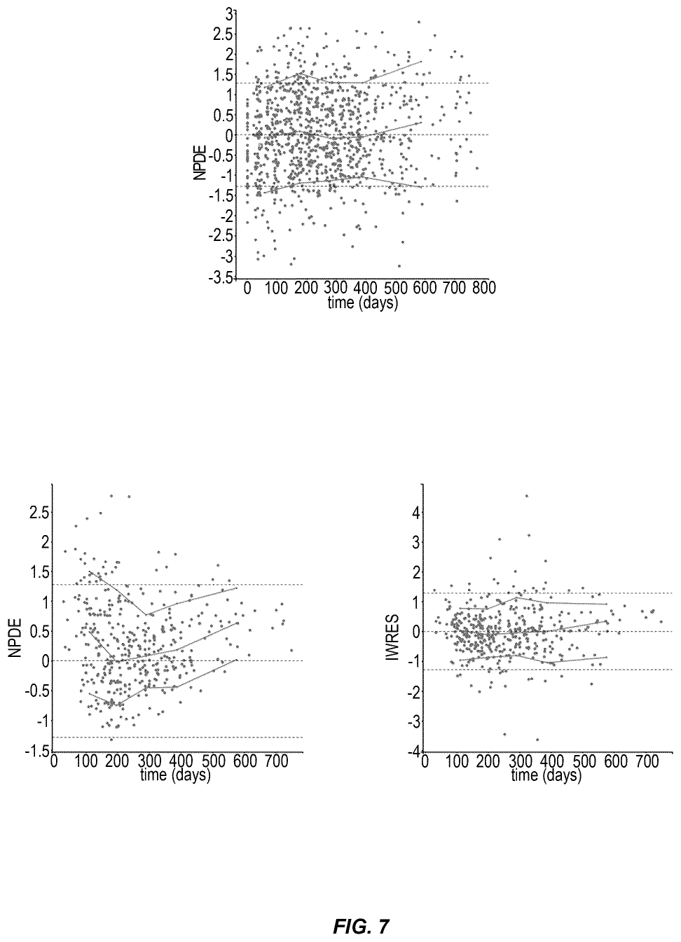

The parameters Amyloid₀, αᵣₑₘₒᵥₐₗ and kᵣₑₚₐᵢᵣ were assumed to have log-normal inter-individual variability and their typical value (fixed effect) and the width of the distribution (random effect) were estimated. The parameters β₁, VWD₅₀ and pow were estimated without any inter-individual variability (i.e. as fixed effects only). It can be shown that VWD(t) is proportional to Amyloid₀, implying that the parameters Amyloid₀ and EG₅₀ effectively appear only as a ratio in the expression for BGTS(t). Therefore, to ensure structural identifiability, EG₅₀ was set to one, without any loss of generality. Moreover, the value of BGTSₘₐₓ was fixed to 60, in line with the limit of the 60-point BGTS severity scale. The residual (unexplained) variability that describes the difference between observed and predicted BGTS was assumed to be independent of the predictions and to be normally distributed. The estimated population parameters (typical values and inter-individual variability) are shown in Table 2. The diagnostic plots from FIG. 7 demonstrate the ability of the model to predict the probability of an ARIA-E event at the population level, as well as the magnitude of ARIA-E, in particular at the individual level: the residuals over time are evenly centered around zero, without a major systemic bias and with most values within −2 and +2 standard deviations.

TABLE 2
Estimates of the population parameters

| | | | |
|---|---|---|---|
| | | | Relative |
| | | | Standard |
| Parameter | Unit | Value | Error (%) |

Fixed effects

| | | | |
|---|---|---|---|
| Amyloid₀ | arbitrary | 3.20 | 6.48 |
| αᵣₑₘₒᵥₐₗ | ${( day \cdot \frac{mcg}{ml} )}^{- 1}$ | 0.126e−3 | 8.75 |
| kᵣₑₚₐᵢᵣ | day⁻¹ | 12.4e−3 | 6.64 |
| β₁ | arbitrary | 9.37 | 8.21 |
| VWD₅₀ | arbitrary | 0.418 | 3.89 |
| EG50 (fixed) | arbitrary | 1.00 | |
| pow | — | 3.72 | 5.5 |
| BGTSₘₐₓ (fixed) | — | 60 | |

Standard deviations of the random effects

| | | | |
|---|---|---|---|
| ω_{Amyloid}_{₀} | arbitrary | 0.405 | 12.9 |
| ω_{α}_{ᵣₑₘₒᵥₐₗ} | ${( day \cdot \frac{mcg}{ml} )}^{- 1}$ | 0.655 | 9.45 |
| ωₖ_{ᵣₑₚₐᵢᵣ} | day⁻¹ | 0.446 | 12.1 |

Standard deviation of the residual error

| | | | |
|---|---|---|---|
| a | — | 2.83 | 4.63 |

The estimated time course for the active-ingredient concentration was calculated using the pharmacokinetics model disclose by Retout S, et al. “Disease Modeling and Model-Based Meta-Analyses to Define a New Direction for a Phase III Program of Gantenerumab in Alzheimer's Disease.” Clin Pharmacol Ther. 2022 April; 111(4):857-866. The estimated time course for local amyloid beta was calculated using Eqn. (1). The estimated time course for VWD was calculated using Eqn. (2). The estimated time course for. BGTS was calculated using Eqns. (3) and (4).

The estimated time courses for the concentrations, local amyloid beta, VWD and BGTS are shown in the middle panel of FIG. 3 for Case 1 and on the left side of FIG. 4 for Cases 2-4.

Cases 1 and 2 had been assigned to the same fast up-titration regimen (regimen 4) but, nonetheless, developed ARIA-E at significantly different times. (See the middle panel of FIG. 3 and the top graph set of FIG. 4). Case 1 reached the target dose by week 16 and experienced the first ARIA-E event at week 20, whereas case 2 developed ARIA-E at week 12 before reaching the target dose. The two cases appear to experience different rates of amyloid removal around week 12 (as can be seen from the different slopes of the local amyloid curves). This is explained in the model by the different estimates of the removal constant (see αᵣₑₘₒᵥₐₗ values in the top right plot of FIG. 4). This difference arises despite similar drug concentration levels during the first 12 weeks and comparable estimates of baseline local amyloid levels.

Case 3, assigned to the slowest up-titration regimen (regimen 1), experienced treatment interruption on two occasions due to ARIA-E. (See the plus symbols in the top graph of the middle plot set of FIG. 4). The development of ARIA-E during slow up-titration may be ascribed to the relatively high baseline level of local amyloid estimated for this subject (see Amyloid₀ values in the middle right distribution plot in FIG. 4). The model results suggested the re-occurrence of ARIA-E upon re-dosing and up-titration by the combination of persisting high levels of local amyloid and relatively high drug concentrations, which drives another rise in VWD.

Case 4, assigned to the second slowest up-titration regimen (regimen 2), developed an early ARIA-E that was much higher in magnitude than any of the ARIA-E events experienced by the other three cases. (See the bottom graph of the bottom plot set of FIG. 4.) The model did not estimate any significantly different levels of baseline local amyloid or drug concentrations that could explain the observed disparity in ARIA-E magnitude. However, both the removal and repair constants estimated for case 4 are significantly different from the constants estimated for the other three cases. Specifically, the strikingly large value of the removal constant drives local amyloid removal at a high rate that cannot be matched by the slow rate of vascular repair determined by the small repair rate constant (see αᵣₑₘₒᵥₐₗ and kᵣₑₚₐᵢᵣ values in FIG. 4). This imbalance leads to a large VWD level and, ultimately, to a high BGTS.

The model predictions for the four ARIA-E cases share multiple features. The model predicted the steepest decrease in local amyloid level and a subsequent increase in VWD and BGTS magnitude around the time of observed ARIA-E events. This pattern is noticeable at week 20 for case 1, week 12 for case 2, weeks 28 and 60 for case 3 and weeks 12 and 52 for case 4 (FIGS. 3 and 7). During the entire period of treatment interruption, the model predicted a plateau in the local amyloid level and a progressive decrease in VWD. The latter also reflects the repair process and translates into a progressive decrease in ARIA-E magnitude that closely follows the observed ARIA-E resolution, as it can be seen during weeks 20 to 36 for case 1, weeks 12 to 24 for case 2, weeks 28 to 44 for case 3 and weeks 12 to 40 for case 4 (FIGS. 3 and 7).

### Exploration of the Influence of Key Model Parameters in the Dynamics of ARIA-E

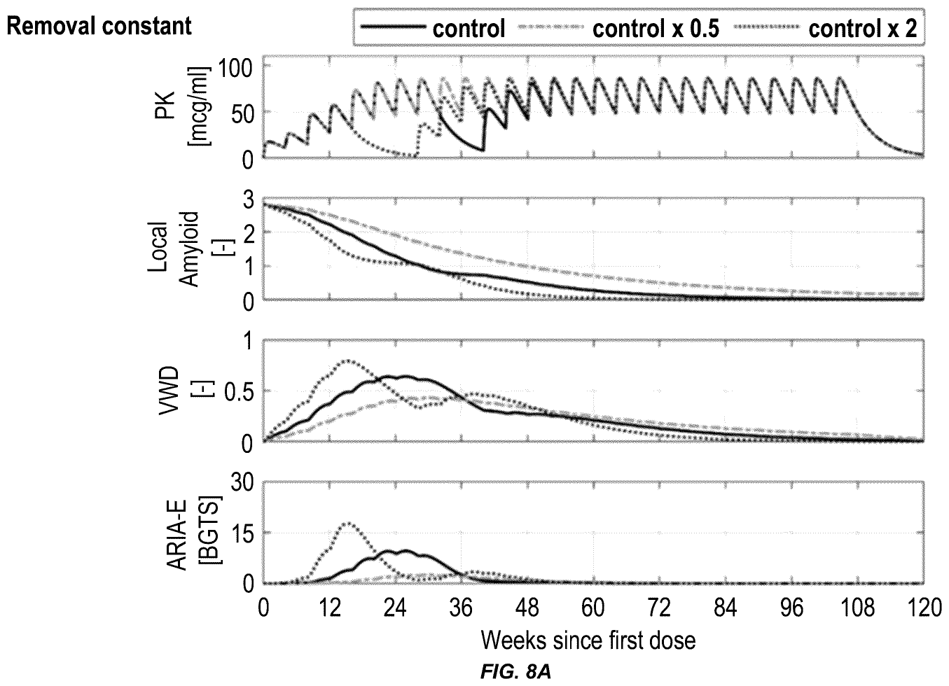

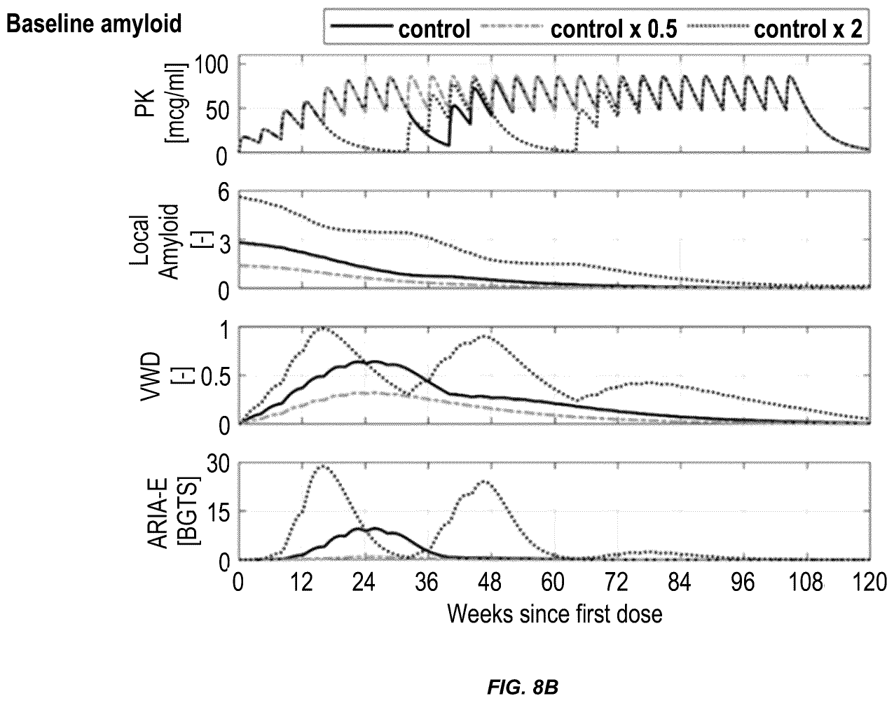

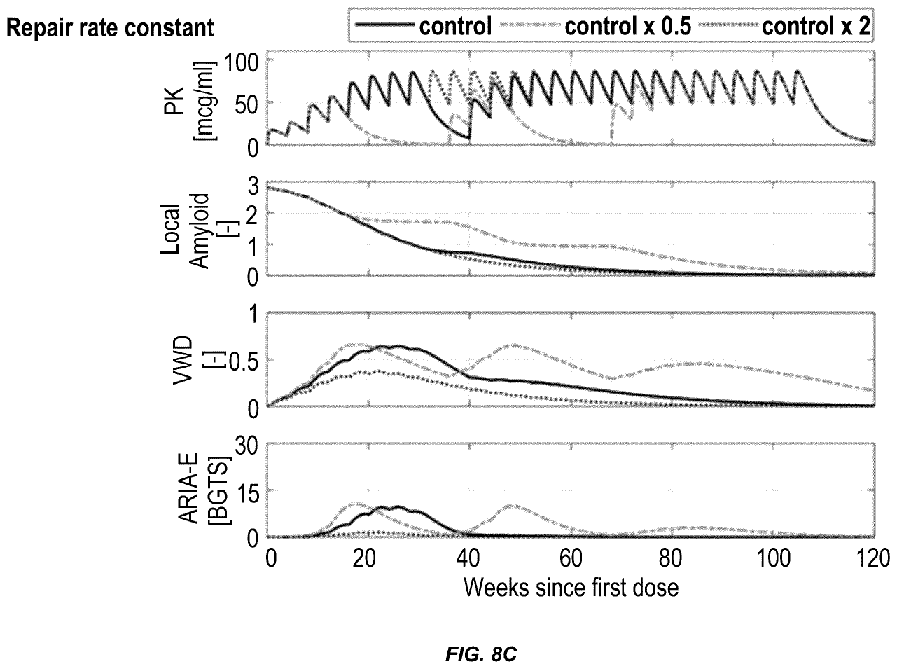

Considering that both the baseline level of local amyloid and the removal constant influence the rate of local amyloid removal, their role in the dynamics of ARIA-E was explored separately in a set of simulations shown in FIGS. 8A-8B. The control plots are those shown in the center panel of FIG. 3, corresponding to case 1. The other plots are simulations where a single variable was modified (the removal constant for FIG. 8A, baseline local amyloid beta for FIG. 8B, and the repair rate constant for FIG. 8C).

The larger removal constant led to earlier increases in both VWD and ARIA-E, while the smaller removal constant delayed the appearance of ARIA-E, but it also slowed the clearance of local amyloid. Data points at weeks 12 and 24 provide a comparison. In the case of a higher baseline level of local amyloid, the model predicted earlier disturbances in the vascular wall and recurrent ARIA-E events due to persisting high levels of local amyloid. In contrast, in the case of a smaller baseline level of local amyloid, the model predicted continuous removal of local amyloid without any significant ARIA-E events. FIG. 8C illustrates the evolution of ARIA-E when the presumed vascular repair process is reduced or intensified through the variation of the repair rate constant. If the intrinsic vascular repair process had a smaller repair rate constant, the removal of local amyloid would result in repeated disturbances in the vascular wall, ultimately leading to ARIA-E recurrence. Conversely, in this model, vascular repair processes with a larger rate constant would be able to counteract the rate of amyloid removal predicted at the end of up-titration and prevent any significant ARIA-E.

### Interpretations

This semi-mechanistic, in silica model tackled the ambiguity and complexity of ARIA-E pathomechanisms in the context of longitudinal observations of ARIA-E magnitude from anti-amyloid clinical trials. Earlier ARIA-E models developed with bapineuzumab and gantenerumab data are event hazard models that can predict the probability of ARIA-E occurrence for various dosing regimens. The mathematical framework used in this Example permits different biological interpretations of the modeled mechanisms, based on the following key factors: local amyloid load, drug exposure (dose and drug concentration), amyloid removal, as well as damage and repair of the vascular wall. For the various ARIA-E scenarios depicted in FIG. 1, the same mathematical relationship describes the drug-mediated removal of local amyloid, whether it is the parenchymal or vascular Aβ burden that is being cleared via direct dissolution or cell-mediated phagocytosis. While the precise biological details underlying ARIA-E remain to be unveiled, the common feature across the different mechanistic pathways that can set off an ARIA-E event seems to be a damaged vascular wall. The model that integrates links drug-mediated removal of local amyloid to the level of VWD, ultimately, to the severity of ARIA-E (as measured by BGTS) performed well in capturing the inter-subject variability. The parameters were estimated with the non-linear mixed effects method by fitting the model to the BGTS dataset from the SR/MR OLE studies of gantenerumab. The resulting VWD model provides a good subject-level description of the ARIA-E reported in the SR/MR OLE studies of gantenerumab, thereby confirming the plausibility of the implemented mechanisms.

Beyond fitting individual time-courses of ARIA-E events, the VWD model offers a number of insights. According to the model, high rates of local amyloid removal can prompt the onset of ARIA-E due to (i) high exposure (dose and drug concentration), (ii) high local amyloid and/or (iii) high efficiency in local amyloid removal (here, captured by the removal constant αᵣₑₘₒᵥₐₗ). For anti-amyloid antibodies, such as aducanumab, donanemab and gantenerumab, which rely heavily on Fc receptor-mediated phagocytosis of amyloid aggregates by immune cells, the higher estimates of the removal constant could reflect a larger, likely exaggerated, response of the immune cells activated by the amyloid-drug complex. An image-based analysis of a small number of ARIA-E cases from the gantenerumab studies revealed that the regions with FLAIR hyperintensities often displayed prominent decreases in the amyloid PET signal, thereby supporting the idea of more efficient cell-mediated phagocytosis of Aβ aggregates at the ARIA-E sites. Noteworthy, significant amyloid reductions were also observed in regions without any FLAIR abnormalities. Based on the VWD model, focal amyloid reductions can occur without ARIA-E as long as the local rates of amyloid removal are counterbalanced by the rate of an intrinsic vascular repair process. Moreover, according to the model, ARIA-E can fully resolve (BGTS=0) before the VWD level returns to zero. This behavior is a consequence of the presumed nonlinear VWD-BGTS relationship, which ensures that small disturbances in the vascular wall do not result in a detectable ARIA-E event, as shown in FIG. 3. Lastly, the VWD model suggests that the reoccurrence of ARIA-E could be a combined effect of high drug concentrations and persisting high levels of local amyloid and that amyloid depletion over time ultimately reduces the risk of ARIA-E, even at high drug concentrations.

When comparing the model results to amyloid reduction observations in anti-amyloid clinical trials that reported ARIA, it is useful to distinguish between the global and local nature of the modeled/measured amyloid. For instance, studies with global PET assessments of baseline amyloid in the bapineuzumab and gantenerumab studies have indicated that there are no any significant differences between the ARIA-E and non-ARIA-E groups. When instead the amyloid PET signal was assessed regionally, the baseline amyloid load from the occipital brain region has been found to be significantly higher in the ARIA-E group than in the non-ARIA-E group. Accounting for the predilection of CAA for the occipital vasculature, the higher baseline levels of local amyloid estimated by the VWD model could be associated with a higher local burden of vascular amyloid.

Based on PET imaging alone, one cannot say with certainty whether the reduction in amyloid PET signal is due to the removal of vascular and/or parenchymal amyloid. The relative contribution of the two forms of Aβ aggregates to the amyloid PET signal has been a matter of debate. Some studies have concluded that amyloid PET can detect vascular Aβ in CAA, while other recent studies reported that the amyloid PET signal is largely driven by parenchymal amyloid plaques and does not appear to be significantly confounded by CAA. The latter findings are limited by the fact that only half of the subjects met the clinical diagnosis of probable CAA with a median number of two cerebral microbleeds, despite the pathological confirmation of CAA in all subjects. Of note, a decade ago, the Alzheimer's Association Research Roundtable Workgroup recommended that individuals with more than four cerebral microhemorrhages at baseline should not be included in anti-amyloid clinical trials. Therefore, the contribution of vascular Aβ aggregates to the PET signal in clinically more severe CAA cases, in particular in those enrolled in more recent anti-amyloid studies, remains to be determined. In order to further clarify the relative risk of ARIA-E associated with the regional load of vascular amyloid and its removal, more analyses that integrate longitudinal PET and MRI data from anti-amyloid trials and quantify the regional distribution of the amyloid signal FLAIR hyperintensities and CAA imaging markers are needed. Future biomarkers that are able to better distinguish between the vascular and parenchymal amyloid burden can be used to refine the VWD model, which could then predict the evolution of ARIA-E in patients with various degrees of AD pathology (based on the parenchymal amyloid burden) and CAA (based on the vascular amyloid burden).

The semi-mechanistic VWD model provides a mathematical and semi-mechanistic explanation of the observed temporal patterns in the BGTS data of gantenerumab studies. The VWD model allows in silica individual-level exploration of biologically plausible variables and processes that influence the development and severity of ARIA-E. Upon its validation, the VWD model will be useful for the generation of hypotheses that can be tested in clinical studies for ARIA management. The VWD model can be used to simulate the impact of the rate of local amyloid removal and vascular repair on the time-course of ARIA-E in individuals with an ARIA-E history. Such model-based predictions could support decisions on the continuation or re-introduction of treatment in order to minimize the risk of ARIA-E progression.

While the present subject matter has been, described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A computer-implemented method comprising:
identifying a dosage of an active ingredient in a treatment that is being provided to or that is being considered for provision to a subject;
predicting a level of local amyloid beta based on the dosage of the active ingredient;
predicting a severity of amyloid-related imaging abnormalities (ARIA) manifested as hyperintensities on T₂-weighted fluid attenuated inversion recovery (FLAIR) images (ARIA-E) based on the predicted removal of local amyloid beta, wherein predicting severity of ARIA-E includes predicting an extent of vascular wall disturbance; and
outputting a result corresponding to the predicted ARIA-E severity.

2. The computer-implemented method of claim 1, wherein the active ingredient includes an anti-amyloid monoclonal antibody.

3. The computer-implemented method of claim 1, further comprising using a pharmacokinetic model to predict a time course of a concentration of an active ingredient in the treatment in plasma, wherein the prediction of the local amyloid beta is based on at least one predicted concentration of the active ingredient in the predicted time course.

4. The computer-implemented method of claim 1, wherein predicting the level of local amyloid beta includes:
estimating a baseline level of local amyloid beta;
calculating a rate of removal of the local amyloid beta based on the dosage of an active ingredient and the baseline level of local amyloid beta; and
predicting the level of local amyloid beta based on the calculated rate of removal of the local amyloid beta.

5. The computer-implemented method of claim 1, wherein predicting the level of local amyloid beta includes solving a pharmacodynamic differential equation that assumes a rate of change of amyloid beta is proportional to a product between concentrations of the active ingredient in the subject and local amyloid beta levels.

6. The computer-implemented method of claim 1, wherein predicting the level of vascular wall disturbance includes solving a differential equation that includes the level of local amyloid beta.

7. The computer-implemented method of claim 1, wherein predicting the severity of ARIA-E includes solving an algebraic equation that assumes a non-linear relationship between the level of vascular wall disturbance and the Barkhof Grand Total Score (BGTS).

8. The computer-implemented method of claim 1, further comprising:
identifying a potential schedule for monitoring for ARIA events based on the predicted severity of ARIA, wherein the result characterizes the potential schedule.

9. The computer-implemented method of claim 1, further comprising:
identifying a potential recommendation of the dosage of the active ingredient for the subject based on the predicted severity of ARIA, wherein the result characterizes the potential recommendation dosage.

10. A system comprising:
one or more data processors; and
a non-transitory computer readable storage medium containing instructions which, when executed on the one or more data processors, cause the one or more data processors to perform a set of actions including:
identifying a dosage of an active ingredient in a treatment that is being provided to or that is being considered for provision to a subject;
predicting a level of local amyloid beta based on the dosage of the active ingredient;
predicting a severity of amyloid-related imaging abnormalities (ARIA) manifested as hyperintensities on T₂-weighted fluid attenuated inversion recovery (FLAIR) images (ARIA-E) based on the predicted removal of local amyloid beta, wherein predicting severity of ARIA-E includes predicting an extent of vascular wall disturbance; and
outputting a result corresponding to the predicted ARIA-E severity.

11. The system of claim 10, wherein the active ingredient includes an anti-amyloid monoclonal antibody.

12. The system of claim 10, wherein the set of actions further comprises using a pharmacokinetic model to predict, a time course of a concentration of an active ingredient in the treatment in plasma, wherein the prediction of the local amyloid beta is based on at least one predicted concentration of the active ingredient in the predicted time course.

13. The system of claim 10, wherein predicting the level of local amyloid beta includes:
estimating a baseline level of local amyloid beta;
calculating a rate of removal of the local amyloid beta based on the dosage of an active ingredient and the baseline level of local amyloid beta; and
predicting the level of local amyloid beta based on the calculated rate of removal of the local amyloid beta.

14. The system of claim 10, wherein predicting the level of local amyloid beta includes solving a pharmacodynamic differential equation that assumes a rate of change of amyloid beta is proportional to a product between concentrations of the active ingredient in the subject and local amyloid beta levels.

15. The system of claim 10, wherein predicting the level of vascular wall disturbance includes solving a differential equation that includes the level of local amyloid beta.

16. The system of claim 10, wherein predicting the severity of ARIA-E includes solving an algebraic equation that assumes a non-linear relationship between the level of vascular wall disturbance and the Barkhof Grand Total Score (BGTS).

17. The system of claim 10, wherein the set of actions further comprises:
identifying a potential schedule for monitoring for ARIA events based on the predicted severity of ARIA, wherein the result characterizes the potential schedule.

18. The system of claim 10, wherein the set of actions further comprises:
identifying, a potential recommendation of the dosage of the active ingredient for the subject based on the predicted severity of ARIA, wherein the result characterizes the potential recommendation dosage.

19. A computer-program product, tangibly embodied in a non-transitory machine-readable storage medium, including instructions configured to cause one or more data processors to perform a set of actions comprising:
identifying, a dosage of an active ingredient in a treatment that is being provided to or that is being considered for provision to a subject;
predicting a level of local amyloid beta based on the dosage of the active ingredient;
predicting a severity of amyloid-related imaging abnormalities (ARIA) manifested as hyperintensities on T₂-weighted fluid attenuated inversion recovery (FLAIR) images (ARIA-E) based on the predicted removal of local amyloid beta, wherein predicting severity of ARIA-E includes predicting an extent, of vascular wall disturbance; and
outputting a result corresponding to the predicted ARIA-E severity.

20. The computer-program product of claim 19, wherein the active ingredient includes an anti-amyloid monoclonal antibody.
